(19) United States
(12) Patent Application Publication
(10) Pub. No.: US 2004/0043938 A1
(43) Pub. Date: Mar. 4, 2004

(54) Combination therapy for estrogen-dependent disorders

(76) Inventor: Dinesh Purandare, Branchburg, NJ (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/416,844

(22) PCT Filed: Nov. 6, 2001
(86) PCT No.: PCT/US01/43847

(51) Int. Cl. A61K 38/09; A61K 31/56; A61K 31/4196; A61K 31/4178

(52) U.S. Cl. 514/16; 514/17; 514/171; 514/383; 514/397

(57) ABSTRACT

The present invention relates to a combination therapy for treating estrogen dependent cancers in susceptible mammals, including humans, comprising the steps of inhibiting hormone output of their testis or ovaries, respectively, and administering to said mammal at least one aromatase inhibitor.

### FIELD OF THE INVENTION

The present invention relates to a combination therapy for treating estrogen dependent cancers in susceptible mammals, including humans, comprising the steps of inhibiting hormone output of their testis or ovaries, respectively, and administering to said mammal at least one aromatase inhibitor.

### BACKGROUND OF THE INVENTION

Various investigators have been studying hormone dependent breast and endometrial cancer. A known form of endocrine therapy in pre-menopausal women is oophorectomy, most commonly performed by surgery or irradiation, two procedures giving irreversible castration. A reversible form oophorectomy of has been achieved by utilizing Luteinizing Hormone Releasing Hormone agonists (“LHRH agonists”) which, following inhibition of secretion of Luteinizing Hormone (“LH”) by the pituitary gland, decrease serum estrogens to castrated levels (Nicholson et al., Brit. J. Cancer 39, 268-273, 1979).

Several studies show that treatment of pre-menopausal breast cancer patients with LHRH agonists induces responses comparable to those achieved with other forms of castration (Klijn et al., J. Steroid Biochem. 20, 1381, 1984; Manni et al., Endocr. Rev. 7: 89-94; 1986).

U.S. Pat. No. 4,775,660 relates to the treatment of female breast cancer by use of a combination therapy comprising administering an antiandrogen and an antiestrogen to a female after the hormone output of her ovaries has been blocked by chemical or surgical means.

U.S. Pat. No. 4,775,661 relates to the treatment of female breast cancer by use of a therapy comprising administering to a female, after the hormone output of her ovaries has been blocked by chemical or surgical means, an antiandrogen and optionally an inhibitor of sex steroid biosynthesis.

U.S. Pat. No. 4,760,053 describes the treatment of selected sex steroid dependent cancers which combines a LHRH agonist and/or an antiandrogen and/or an antiestrogen and/or at least one inhibitor of sex steroid biosynthesis.

In U.S. Pat. No. 4,472,382 it is disclosed that prostatic adenocarcinoma, benign prostatic hypertrophy and hormone-dependent mammary tumors may be treated with various LH-RH agonists and that prostate adenocarcinoma and benign hypertrophy may be treated by use of various LHRH agonists and an antiandrogen.

U.S. Pat. No. 5,550,107 relates to a treatment of female breast and endometrial cancer by use of a therapy comprising administering to a female after the hormone output of the ovaries has been blocked an antiestrogen and at least one compound selected, e.g., from an androgen, a progestin, at least one inhibitor of sex steroid biosynthesis and one inhibitor of prolactin secretion.

Some clinical improvement in pre-menopausal women with breast cancer by use of three LHRH agonists, goserelin, buserelin and leuprolide, is also reported by C. W. Taylor, “Multicenter randomized clinical trial of goserelin vs. surgical ovariectomy in premenopausal patients with receptor positive breast cancer” (J. Clin. Onc. 1998 (16): 994-999), H. A. Harvey et al. “LH-RH analogs in the treatment of human breast cancer”, LHRH and its Analogs—A new Class of contraceptive and therapeutic Agents (B. H. Vickery and J. J. Nestor, J., and E. S. E. Hafez, eds) Lancaster, MTP Press, (1984) and the J. G. M. Klijn et al., “Treatment with luteinizing hormone-relating hormone analogue (Buserelin) in premenopausal patients with metastatic breast cancer”, Lancet 1, 1213-1216 (1982).

Stein R. C. et al. in British Journal of Cancer: 62, 679-683 (1990) describe the clinical and endocrine effects of 4-hydroxyandrostenedione (formestane) alone and in combination with goserelin in premenopausal woman with breast cancer. Celio L. et al. in European Journal of Cancer: S 155, 691 (Sep. 17, 1997) and in Anticancer Research: 19, 2261-268 (1999) describe a study in premenopausal women with breast cancer by treatment with triptorelin and 4-hydroxyandrostenedione. Dowsett M. et al. in Breast Cancer Research and Treatment: 56, 24-35 (1999) describe a combined treatment with vorozole and goserelin of breast cancer in premenopausal women.

Tsuchiya N. et al. in International Journal of Clinical Oncology: (200) 5: 183-187 describe the effects of fadrazole and leuprorelin acetate on cell proliferation in a human breast cancer cell line.

It is an object of the present invention to provide a method for treating estrogen dependent cancers in mammals, in particular sex steroid dependent cancers, said method being not as invasive as surgery.

### DETAILED DESCRIPTION OF THE INVENTION

The invention provides a method for treating an sex-steroid dependent cancer in a mammal in need of such treatment, including humans, comprising administering simultaneously, separately or sequentially to said mammal an aromatase inhibitor and a LHRH agonist or antagonist, in amounts and close in time sufficient to achieve a therapeutically useful effect, and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.

Preferably such human is a premenopausal woman.

The present invention also provides the use of an aromatase inhibitor in the manufacture of a medicament for treating a sex steroid dependent cancer in a mammal, including humans, undergoing a simultaneous, separate or sequential treatment with a LHRH agonist or antagonist, and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.

The invention also provides a product containing an aromatase inhibitor and a LHRH agonist or antagonist as a combined preparation for simultaneous, separate or sequential use in treating sex steroid dependent cancers, and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.

The estrogen-dependent cancers that can by treated by the combined therapy method provided by the present invention are cancers known in the art as “sex steroid dependent cancers”. Examples of such cancers are testicular cancer, prostate cancer, ovarian cancer, pancreatic cancer, uterine cancer, celomic epithelial carcinoma, germ cell ovarian cancer, fallopian tube ovarian cancer, breast cancer and lung cancer.

In one embodiment of the invention, such cancers are prostate cancer, ovarian cancer and breast cancer, in particular breast cancer in a premenopausal woman.

Examples of aromatase inhibitors according to the invention are exemestane, formestane, fadrozole, letrozole, vorozole and anastrozole, preferably exemestane, anastrozole and letrozole, in particular exemestane.

The term “aromatase inhibitor” is meant to comprise both a single aromatase inhibitor or a mixture of two or more, preferably two, aromatase inhibitors as defined above. Preferably the single aromatase inhibitor, or one of the component of the mixture, is exemestane.

Examples of LHRH agonists according to the invention are, e.g., leuprorelin, deslorelin, triptorelin, buserelin, nafarelin, goserelin, avorelin, histerelin, compound PTL 03001 (5-oxo-L-propyl-L-histidyl-L-tryptophyl-L-seryl-L-tyrosyl-D-tryptophyl-L-leucyl-L-arginyl-N-ethyl-L-prolinamide) (Peptech), compound AN 207 (6-[N6-[5-[2-[1,2,3,4,6,11-hexahydro-2,5,12-trihydroxy-7-mehoxy-6,11-dioxo-4-[[2,3,6-trideoxy-3-(2,3-dihydro-1H-pyrrol-1-yl)α-L-lyxo-hexopyranosyl]oxy]-2-naphthacenyl]-1,5-dioxopentyl]-D-lysine]-,(2S-cis)-) (ASTA Medica Inc.), compound AN 238 L-threoninamide, N-[5-[2-[(2S,4S)-1,2,3,4,6,11-hexahydro-2,5,12-trihydroxy-7-methoxy-6,11--dioxo-4-[[2,3,6-trideoxy-3-(2,3-dihydro-1H- pyrrl-1-yl)α-L-lyxo-hexopyranosyl]oxy]-2-naphthacenyl]-2-oxoethoxy]-1,5-dioxopentyl]-D-phenylalanyl-L-cysteinyl-L-tyrosyl-D-tryptophyl-L-lysyl-L-valyl-L-cysteinyl-, cyclic (2 7)-disulfide (ASTA Medica Inc.) and compound SPD 424 (LHRH-hydrogel implant) (Shire Pharmaceuticals Group), or a pharmaceutically acceptable salt thereof

In one embodiment of the invention, LHRH agonists are triptorelin and goserelin, or a pharmaceutically acceptable salt thereof, in particular triptorelin or a pharmaceutically acceptable salt thereof.

Examples of LHRH antagonists, according to the invention, are e.g. cetrorelix, abarelix, ramorelix, teverelix, ganirelix, compounds A 75998 (Acetyl-D-(2-naphthyl)alanyl-D-(4-chlorophenyl)alanyl-D-(3-pyridyl)alanyl-seryl-(N-methyl)tyrosyl-N6-(nicotinoyl)-D-lysyl-leucyl-N6-(isopropyl)lysyl-propyl-D-alaninamide) and A 84861 (Tetrahydrofuran-2-(S)-ylcarbonyl-glycyl-D-(2-naphthyl)alanyl-D-(4-cholro)phenylalanyl-D-(3-pyridyl)-anayl-L-(N-methyl)tyrosyl-D-[N6-(3-pyridylcarbonyl)]lysyl-L-leucyl-L-(N6-isopropyllysyl-L-propyl-D-alanylamide)(Abbot Labs.), GnRH immunogen (Aphton Co.), compound T 98475 (Isopropyl 3-(N-benzyl-N-methylaminomethyl)-7-(2,6-difluorobenzyl)-4,7-dihydro-2-(4-isobutyrylaminophenyl)-4-oxothieno[2,3-bpyridine-5-carboxylate hydrochloride) (Takeda), and compound MI 1544 (Acetyl-D-tryptophyl-D-cyclopropyl-alanyl-D-tryptophyl-L-seryl-L-tyrosyl-D-lysyl-L-leucyl-L-arginyl-L-propyl-D-alaninamide), or a pharmaceutically acceptable salt thereof An exemplary LHRH antagonist is abarelix or a pharmaceutically acceptable salt thereof.

The inventors of the present invention have also found that treatment of the above mentioned sex steroid-dependent disorders by combined administration of a therapeutically effective amount of an aromatase inhibitor and a therapeutically effective amount of a LHRH agonist or antagonist, can produce a therapeutic effect which is greater than that obtainable by single administration of a therapeutically effective amount of sole LHRH agonist or antagonist.

Most importantly, they have found that such newly obtained therapeutic effect is not paralleled by the toxic effects, otherwise caused by single administration of either therapeutically effective amounts of an aromatase inhibitor or, of the LHRH agonist or antagonist.

As used herein, the term “treating” means in particular “controlling the growth” of the neoplasm, namely slowing, interrupting, arresting, stopping or reversing the neoplasm formation and it does not necessarily indicate a total elimination of the neoplasm.

Therefore, the term “therapeutically useful effect”, besides slowing, interrupting, arresting, stopping or reversing, the neoplasm formation, simply also means that the life expectancy of an individual affected with a cancer will be increased, that one or more of the symptoms of the disease will be reduced and/or that quality of life will be enhanced.

Method and Administration

In effecting treatment of a patient in a therapy method according to the invention, the aromatase inhibitor and the LHRH agonist or antagonist can be administered in any form or mode which makes the compounds bioavailable in effective amounts, including oral and parenteral routes.

By the term “administered” or “administering” as used herein is meant any acceptable manner of administering a drug to a patient which is medically acceptable including parenteral and oral administration.

By “parenteral” is meant intravenous, subcutaneous, intradermal or intramuscular administration.

Oral administration includes administering one or both of the constituents of the combined preparation in a suitable oral form such as, e.g., tablets, capsules, suspensions, solutions, emulsions, powders, syrups and the like.

The actual preferred method and order of administration of the combined preparations of the invention can vary according to, inter alia, the particular pharmaceutical formulation of the aromatase inhibitor being utilized, the particular pharmaceutical formulation of the LHRH agonist or antagonist being utilized, the particular sex steroid-dependent cancer to be treated and the particular patient being treated.

The term “close in time” means that in the combined method of treatment according to the subject invention, the aromatase inhibitor can be administered simultaneously with the LHRH agonist or antagonist or the compounds can be administered sequentially, in either order. However, the compounds are administered in such a way that both inhibition of hormone output of mammal's testis or ovaries and inhibition of aromatase enzyme are contemporaneously provided, and thus a therapeutically useful effect is achieved.

Dosage

The dosage ranges for the administration of the combined preparation can vary with the age, condition and extent of the disease in the patient and can be determined by one of skill in the art.

The dosage regimen must therefore be tailored to the particular of the patient's conditions, response and associated treatments in a manner which is conventional for any therapy, and can be adjusted in response to changes in conditions and/or in light of other clinical conditions.

An effective amount of an aromatase inhibitor antitumor agent can vary from about 0.5 to about 500 mg per dose 1-2 times a day.

Fadrozole, for example, can be administered orally in a dosage range varying from about 0.5 to about 10 mg, and particularly, from about 1 to about 2 mg. Letrozole, for example, can be administered orally in a dosage range varying from about 0.5 to about 10 mg, and particularly, from about 1 to about 2.5 mg. Formestane, for example, can be administered parenterally in a dosage range varying from about 250 to about 500 mg, and particularly, from about 250 to about 300 mg. Anastrozole, for example, can be administered orally in a dosage range varying from about 0.5 to about 10 mg, and particularly, from about 1 to about 2 mg. Exemestane for instance can be administered orally in a dosage range varying from about 5 mg daily to about 600 mg daily, in particular from about 10 to about 50, more particularly from about 10 to about 25 mg daily, or parenterally in a dosage ranging from about 50 to about 500 mg per injection.

An effective amount of LHRH agonist or antagonist is in general the one commonly used in therapy for such compounds. Goserelin can be administered as goserelin acetate by subcutaneous administration of slow release goserelin at a dosage from about 3 to about 12 mg. Triptorelin can be administered for instance as triptorelin pamaote by intramuscular administration in the form of a depot formulation at a dosage from about 3 to about 20 mg, in such a way that there is an interval of about 1, 2, 3 or 4 months between each administration. In particular triptorelin pamoate can be administered intramuscularly in the form of microparticles as described in U.S. Pat. Nos. 5,225,205 and 5,776,885, and more specifically as 1-month depot formulation 3.75 mg. For instance, abarelix can be administered as single intramuscular administration of slow release abarelix 10 to 200 mg every 2 weeks or every month.

The invention provides a method of treating a sex steroid dependent cancer selected from ovarian and breast cancer in a pre-menopausal woman in need of such treatment, comprising administering substantially simultaneously to said woman exemestane and triptorelin or a pharmaceutically acceptable salt thereof, in amounts and close in time sufficient to achieve a therapeutically useful effect.

The term “substantially simultaneous” means that exemestane and triptorelin are administered in such a way that both inhibition of hormone out-put of her ovaries and inhibition of aromatase enzyme are contemporaneously provided, and thus a therapeutically useful effect is achieved.

As a further embodiment of the invention it is here also provided the use of exemestane in the manufacture of a medicament for treating a sex steroid dependent cancer selected from ovarian and breast cancer in premenopausal woman, undergoing a substantially simultaneous treatment with triptorelin or a pharmaceutically acceptable salt thereof In one embodiment of the invention breast cancer is treated.

In one embodiment of the invention, exemestane and triptorelin, in particular as pamoate salt, are administered substantially simultaneously, as herein described, to achieve a therapeutically useful effect.

In particular, triptorelin pamoate can be administered as a sustained release formulation, in such a way that there is an interval from about 1 to 4 months between each administration, e.g. in the form of 1 month depot 3.75 mg formulation, as described in U.S. Pat. Nos. 5,225,205 and 5,776,885. Exemestane can be administered parenterally at a dosage ranging from about 50 to about 500 mg per injection, or orally at a dosage from about 10 to about 25 mg daily.

As stated above, the invention also provides kits or single packages containing the pharmaceutical compositions useful for the combination treatment of the selected sex steroid-dependent cancers discussed above. The kits or packages can also contain instructions to use the pharmaceutical compositions in accordance with the present invention.

As an example a kit according to the present invention provides an exemestane 25 mg oral or 50-500 mg parenteral composition and a triptorelin 1 month depot formulation 3.75 mg.

A pharmaceutical composition for intramuscular administration containing triptorelin pamoate in the form of a depot formulation can be prepared as described in U.S. Pat. Nos. 5,225,205 and 5,776,885.

A pharmaceutical composition containing exemestane can be prepared, for example, according to U.S. Pat. No. 4,808,616.

All references cited in this disclosure are incorporated herein by reference.

1. A method for treating a sex steroid dependent cancer in a mammal in need of such treatment, comprising administering simultaneously, separately or sequentially to said mammal an aromatase inhibitor and a LHRH agonist or antagonist, in amounts sufficient to achieve a therapeutically useful effect and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.

2. The method according to claim 1, wherein the sex steroid dependent cancer is selected from the group consisting of testicular cancer, prostate cancer, ovarian cancer, pancreatic cancer, uterine cancer, celomic epithelial carcinoma, germ cell ovarian cancer, fallopian tube ovarian cancer, breast cancer and lung cancer.

3. The method according to claim 1, wherein the estrogen-dependent cancer is breast cancer in a premenopausal woman.

4. The method of claim 1, wherein the mammal is a human.

5. The method according to claim 1, wherein the aromatase inhibitor is selected from the group consisting of exemestane, formestane, fadrozole, letrozole, vorozole, anastrozole, and a mixture of two or more of them.

6. The method according to claim 1, wherein the aromatase inhibitor is exemestane.

7. The method according to claim 5, wherein when administered orally, the amount of aromatase inhibitor exemestane is from about 5 to about 600 mg, fadrozole from about 0.5 to about 10 mg, letrozole from about 0.5 to about 10 mg, and anastrozole from about 0.5 to about 10 mg.

8. The method according to claim 5, wherein when administered parenterally, the amount of aromatase inhibitor exemestane is from about 5 to about 500 mg, and formestane is from about 250 to about 500 mg.

9. The method according to claim 1, wherein the LHRH agonist is selected from the group consisting of leuprorelin, deslorelin, triptorelin, buserelin, nafarelin, goserelin, avorelin, histerelin, compound PTL 03001 (5-oxo-L-propyl-L-histidyl-L-tryptophyl-L-seryl-L-tyrosyl-D-tryptophyl-L-leucyl-L-arginyl-N-ethyl-L-prolinamide), compound AN 207 (6-[N6-[5-[2-[1,2,3,4,6,11-hexahydro-2,5,12-trihydroxy-7-mehoxy-6,11-dioxo-4-[[2,3,6-trideoxy-3-(2,3-dihydro-1H-pyrrol-1-yl)α-L-lyxo-hexopyranosyl]oxy]-2-naphthacenyl]-1,5-dioxopentyl]-D-lysine]-,(2S-cis)-), compound AN 238 (L-threoninamide, N-[5-[2-[(2S,4S)-1,2,3,4,6,11-hexahydro-2,5,12-trihydroxy-7-methoxy-6,11-dioxo-4-[[2,3,6-trideoxy-3-(2,3-dihydro-1H-pyrrl-1-yl)α-L-lyxo-hexopyranosyl]oxy]-2-naphthacenyl]-2-oxoethoxy]-1,5-dioxopentyl]-D-phenylalanyl-L-cysteinyl-L-tyrosyl-D-trypphyl-L-lysyl-L-valyl-L-cysteinyl-, cyclic(2 7)-disulfide), compound SPD 424 (LHRH-hydrogel implant), and a pharmaceutically acceptable salt thereof.

10. The method according to claim 1, wherein the LHRH agonist is selected from triptorelin, goserelin, and a pharmaceutically acceptable salt thereof.

11. The method according to claim 1, wherein the LHRH agonist is—triptorelin or a pharmaceutically acceptable salt thereof.

12. The method according to claim 1, wherein the LHRH agonist is triptorelin pamoate.

13. The method according to claim 1, wherein the LHRH agonist triptorelin pamoate is in the form of a depot formulation, at a dosage from about 3 to about 20 mg.

14. The method according to claim 13, wherein the LHRH agonist triptorelin pamoate is in the form of a 1 month depot formulation 3.75 mg.

15. The method according to claim 1, wherein the LHRH antagonist is selected from the group consisting of cetrorelix, abarelix, ramorelix, teverelix, ganirelix, compound A 75998 (Acetyl-D-(2-naphthyl)alanyl-D-(4-chlorophenyl)alanyl-D-(3-pyridyl)alanyl-seryl-methyl)tyrosyl-N6-(nicotinoyl)-D-lysyl-leucyl-N6-(isopropyl)lysyl-propyl-D-alaninamide), compound A 84861 (Tetrahydrofuran-2-(S)-ylcarbonyl-glycyl-D-(2naphthyl)alanyl-D-(4-cholro)phenylalanyl-D-(3-pyridyl)-alanyl-L-(N-methyl)tyrosyl[N6-(3-pyridylcarbonyl)]lysyl-L-leucyl-L-(N6-isopropyl)lysyl-L-propyl-D-alanylamide), GNRH immunogen, compound T 98475 (Isopropyl 3-(N-benzyl-N-methylaminomethyl)-7-(2,6-difluorobenzyl)-4,7-dihydro-2-(4-isobutyrylaminophenyl)-4-oxothieno[2,3-bpyridine-5-carboxylate hydrochloride), compound MI 1544 (Acetyl-D-tryptophyl-D-cyclopropyl-alanyl-D-tryptophyl-L-seryl-L-tyrosyl-D-lysyl-L-leucyl-L-arginyl-L-propyl-D-alaninamide), and a pharmaceutically acceptable salt thereof.

16. A method of treating a sex steroid dependent cancer selected from ovarian and breast cancer in a pre-menopausal woman in need of such treatment, comprising administering to said woman exemestane and triptorelin or a pharmaceutically acceptable salt thereof, in amounts sufficient to achieve a therapeutically useful effect.

17. The method according to claim 16, wherein both inhibition of hormone out-put of the women's ovaries and inhibition/inactivation of aromatase enzyme are contemporaneously provided, and thus a therapeutically useful effect is achieved.

18. The method according to claim 16, wherein the estrogen dependent cancer is breast cancer.

19. The method according to claim 16, wherein triptorelin is in the form of triptorelin pamoate salt.

20. The method according to claim 16, wherein triptorelin pamoate is in the form of a depot formulation.

21. The method according to claim 16, wherein triptorelin pamoate is in the form of 1 month depot formulation 3.75 mg.

22. The method according to claim 16, wherein about 5 to 600 mg/day of exemestane is administered orally.

23. The method according to claim 16, wherein about 10 to 500 mg/day of exemestane is administered orally.

24. The method according to claim 16, wherein about 25 mg/day of exemestane is administered orally.

25. The method according to claim 16, wherein about 50 to 500 mg/day of exemestane is administered parenterally.

26. Use of an aromatase inhibitor in the manufacture of a medicament for treating a sex steroid dependent cancer in a mammal undergoing a simultaneous, separate or sequential treatment with a LHRH agonist or antagonist, and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.

27. Use according to claim 26, wherein the mammal is a human.

28. Use according to claim 26, wherein the aromatase inhibitor is exemestane, the LHRH agonist is triptorelin and the sex steroid dependent cancers are ovarian and breast cancers.

29. Product containing an aromatase inhibitor and a LHRH agonist or antagonist as a combined preparation for simultaneous, separate or sequential use in treating sex-dependent cancers, and wherein, when the cancer is breast cancer, and a) the LHRH agonist is triptorelin, then the aromatase inhibitor is other than formestane, b) the LHRH agonist is goserelin, then the aromatase inhibitor is other than vorozole or formestane, or c) the LHRH agonist is leuprorelin, then the aromatase inhibitor is other than fadrozole.
